(19) United States
(12) Patent Application Publication
(10) Pub. No.: US 2023/0127349 A1
(43) Pub. Date: Apr. 27, 2023

(54) Device for Closing a Perforation in a Wall of a Cavity in a Human or Animal Body

(71) Applicant: ASSISTANCE PUBLIQUE HÔPITAUX DE PARIS, Paris (FR)

(72) Inventor: Pierre Mordant, Paris (FR)

(21) Appl. No.: 17/768,433

(22) PCT Filed: Oct. 29, 2020
(86) PCT No.: PCT/EP2020/080373

§ 371 Date: Dec. 21, 2022

(30) Foreign Application Priority Data
Nov. 4, 2019 (FR) 1912333

(51) Int. Cl. A61B 17/00; A61M 1/36

(52) U.S. Cl. CPC A61B 17/0057; A61M 1/3666; A61M 1/3655; A61B 2017/00004; A61B 2017/00867; A61B 2017/00668; A61B 2017/00623

(57) ABSTRACT

The invention relates to a device (10) for closing a perforation (21) in a wall (2), the closing device (10) having closing means (11) comprising:
a central portion (14),
a first end (12) having a first hooking means (12′) and a second end (13) having a second hooking means (13′), the two ends (12,13) being arranged on either side of the central portion (14),
   the closing means (11) being made of a shape memory material and having a structure allowing a variation in the relative position of the hooking means (12′, 13′) with respect to one another, depending on the state of compression of the central portion (14).

### TECHNICAL FIELD OF THE INVENTION

The technical field of the invention is that of medical instruments.

In particular, the invention relates to a device and a method allowing to close a perforation in a cavity in a human or animal body.

Furthermore, the invention relates to a catheter introducer in a cavity in a human or animal body comprising such a closing device. Moreover, the invention relates to an injection or sampling cannula for an extracorporeal membrane oxygenation system comprising such a closing device. In addition, the invention relates to an extracorporeal membrane oxygenation system comprising such a cannula.

### TECHNOLOGICAL BACKGROUND OF THE INVENTION

An extracorporeal membrane oxygenation system, known as ECMO, comprising a set of components allowing to extract, by means of a sampling cannula, a blood volume from a cavity in a human or animal body, to oxygenate it, to decarboxylate it, possibly to heat it, then to reinject it, by means of an injection cannula, into a cavity different from or identical to the sampling cavity.

After using sampling and injection cannulas, it is necessary to ensure the closing of the perforation formed at the insertion point of the cannula in the cavity. Yet, the diameter of the cannulas used in ECMO systems is important, generally between 5 and 8 mm, such that the removal of the cannulas leaves a large opening in the cannulated cavity.

In most cases, the perforation is closed up through open surgery which requires the intervention of a specialist surgeon, access to an operating theatre and a potentially morbid surgical approach (disunity, lymphorrhoea, lymphocele, infection, etc.).

In rarer cases, the perforation is closed up by manual compression. However, if this technique works in 80% of cases, in the remaining 20%, it requires emergency surgery.

In other rarer cases, the perforation is closed up by a percutaneous closing system implemented before the insertion of the cannula or after its removal, which makes it possible to avoid open surgery. However, given the complexity of this technique, only very specialist teams can do it. In addition, these teams must have access to an operating theatre in case of problems.

### SUMMARY OF THE INVENTION

The invention offers a solution to the problems mentioned above, by proposing a device and a method for closing a perforation which is simple to use and which does not require open surgery and therefore does not require making a surgeon intervene or does not require having access to an operating theatre.

A first aspect of the invention relates to a device for closing a perforation in a wall in a human or animal body, said wall having an internal part and an external part, the closing device having closing means comprising:
a central portion,
a first end having a first hooking means and a second end having a second hooking means, the two ends being arranged on either side of the central portion, each hooking means being configured to be anchored on a border of the perforation at the internal part of the wall of the cavity.

In addition, the closing means are made of a shape memory material and have a structure allowing a variation in the relative position of the hooking means with respect to one another according to the state of compression of the central portion such that when the central portion is in a compressed state, the distance between the hooking means increases and that when the central portion passes from a compressed state to a relaxed state, the distance between the hooking means decreases, which leads to the alignment of the borders of the perforation and therefore the closing of the perforation.

By “cavity of the body”, this means an organ, a blood vessel, a lymphatic vessel or also a bronchial structure.

Thanks to the closing device according to the invention, the operation of closing a perforation in a wall of a cavity is simplified. Indeed, it suffices to place the closing means in the cavity, via the perforation, to compress the central portion so as to spread the hooking means such that they can be anchored on the borders of the perforation, at the internal part of the wall of the cavity, then to relax the central portion, in order to reduce the gap between the hooking means which allows to progressively align the borders of the perforation, and therefore to ensure its closing.

Due to its ease of use, the closing device does not require nor perform a surgical intervention and all the more so, the intervention of a specialist surgeon or access to an operating theatre. In addition, the closing means can be left in place in the cavity for a long duration.

Further to the characteristics which have just been mentioned in the preceding paragraph, the closing device according to the first aspect of the invention can have one or more complementary characteristics from among the following, considered individually or according to any technical possible combinations.

According to a non-limiting embodiment, the closing device has a guide channel having an end adapted to be positioned in the cavity, through the perforation, the guide channel being configured to receive the closing means and to ensure the compression of said closing means.

According to a non-limiting embodiment, the closing means are configured to adopt:
a compression position in which the central portion and the hooking means are in the guide channel and in a compressed state,
a rest position in which the central portion and the hooking means are outside of the guide channel and in a relaxed state,
an intermediate position in which the central portion is in a guide channel, in a compressed state, while the hooking means are outside of the guide channel, in a relaxed state, the distance between the hooking means in the intermediate position being greater than the distance between the hooking means in the rest position.

Advantageously, when the closing device is integrated in a cannula, the closing means can, when they are in the intermediate position, have an abutment function allowing the positioning of the cannula and an anchoring function allowing the removal of the cannula during its use.

According to a non-limiting embodiment, the closing device has movement means configured to ensure the movement of the closing means inside the guide channel.

According to a non-limiting embodiment, the movement means are removably fixed to the central portion of the closing means.

According to a non-limiting embodiment, the central portion has the form of a loop.

According to a non-limiting embodiment, the first hooking means and the second hooking means each have the form of a hook.

According to a non-limiting embodiment, the closing means have an antithrombogenic coating in order to prevent the formation of thrombus and/or antiproliferative coating to favour haemostasis at the perforation and to prevent the formation of a hyperplasia of the cavity and/or a prothrombogenic coating to favour haemostasis at the perforation and/or a procoagulant coating.

According to a non-limiting embodiment, the closing means are made of a resorbable material in order to decrease the risk of long-term complications.

According to a non-limiting embodiment, the closing means are made from a nickel-titanium alloy wire.

Furthermore, the invention according to a second aspect relates to an catheter introducer in a cavity of a human or animal body, through a perforation, having a conduit configured for the passage of the catheter, characterised in that it has a closing device according to the first aspect, configured to be positioned inside the conduit and to ensure the closing of the perforation by deploying the closing means of the closing device in the cavity during the removal of the introducer from the perforation.

In addition, the invention according to a third aspect relates to a cannula for injecting or sampling a fluid in a cavity in a human or animal body, through a perforation, having:
a main lumen defining a first space of the cannula for the circulation of the fluid,
a first auxiliary lumen defining a second space of the cannula and having an outlet leading into the cavity,
a first closing device according to the first aspect configured to be positioned inside the first auxiliary lumen and to ensure the closing of the perforation by deploying the closing means of the first closing device in the cavity during the removal of the cannula from the perforation.

Further to the characteristics which have just been mentioned in the preceding paragraph, the cannula according to the third aspect of the invention can have one or more complementary characteristics from among the following, considered individually or according to any technical possible combinations.

According to a non-limiting embodiment, the cannula has:
a second auxiliary lumen defining a third space of the cannula and having an outlet leading into the cavity,
a second closing device according to the first aspect configured to be positioned inside the second auxiliary lumen.

The presence of two closing devices allows to close the perforation more effectively.

According to a non-limiting embodiment, the first auxiliary lumen and/or the second auxiliary lumen is/are arranged in a peripheral portion of the cannula, which can be the wall of the main lumen.

According to a non-limiting embodiment, the closing means form means for anchoring the cannula in the cavity when the closing means are in an intermediate position.

Thus, the closing means allow to block in position the injection cannula in the cavity such that it does not risk being extracted from the body of the patient even in case of movements of the patient. Thus, the injection cannula can be held in the body of a patient for a long duration.

Moreover, the invention according to a fourth aspect relates to an extracorporeal membrane oxygenation system having:
a sampling cannula for receiving a low-oxygen fluid;
a pump for ensuring the pumping of the low-oxygen fluid coming from the sampling cannula;
an oxygenator for ensuring the oxygenation of the low-oxygen fluid coming from an outlet of the pump;
an injection cannula for injecting the oxygenated fluid into the cavity,
   the sampling cannula and/or the injection cannula being formed by the cannula according to the second aspect.

Furthermore, the invention according to a fifth aspect relates to a method for closing a perforation, by means of the closing device having:
a step of inserting the end of the guide channel into the cavity, the closing means are compressed inside the guide channel;
a step of partially deploying the closing means in the cavity, the ends of the closing means are deployed outside of the guide channel while the central portion of the closing means is kept in a compressed state inside the guide channel;
a step where the movement means and the guide channel are partially removed from the cavity, through the perforation, until the hooking means are hooked on the borders of the perforation at the internal part of the wall of the cavity, the central portion of the closing means is kept compressed in the guide channel;
a step where the guide channel is fully removed from the cavity leading to the deployment of the central portion, the hooking means are hooked to the borders of the perforation, at the internal part of the wall of the cavity and form an abutment preventing the closing means from being extracted from the cavity.

The invention and its different applications will be best understood upon reading the following description and upon examining the figures which accompany it.

### BRIEF DESCRIPTION OF THE FIGURES

The figures are presented for information and not at all limiting of the invention.

FIG. 1 illustrates the closing device according to an embodiment of the invention.

FIG. 2a is an enlargement of the closing means of the closing device represented in FIG. 1.

FIG. 2b illustrates the closing means of the closing device represented in FIG. 1, in an intermediate position.

FIG. 2c illustrates the closing means of the closing device represented in FIG. 1, in a compression position.

FIG. 3 is a block diagram illustrating the method for closing a perforation by using the closing device represented in FIG. 1.

FIG. 4 illustrates the closing device represented in FIG. 1, when it is positioned in the perforation and that the closing means are in the compression position.

FIG. 5 illustrates the closing device represented in FIG. 4, when the closing means are in the intermediate position and that they are anchored on the borders of the perforation.

FIG. 6 illustrates the passage from the compression position represented in FIG. 4 to the intermediate position represented in FIG. 5.

FIG. 7 illustrates the passage from the intermediate position represented in FIG. 5 to the rest position and to the closing of the perforation.

FIG. 8 is a schematic representation of an ECMO system according to an embodiment of the invention.

FIG. 9 illustrates an injection cannula according to a first embodiment of the invention, used in the ECMO system represented in FIG. 8.

FIG. 10 is a transverse cross-sectional view of an injection cannula according to a second embodiment.

FIG. 11 shows a side view of a portion of the injection cannula represented in FIG. 10, when the closing means are in the compression position.

FIG. 12 shows a side view of a portion of the injection cannula represented in FIG. 10, when the closing means are in the intermediate position.

The figures are presented for information and not at all limiting of the invention.

### DETAILED DESCRIPTION

The invention relates to a closing device 10 allowing to close a perforation 21 in the wall 2 of a cavity 1 in a human or animal body.

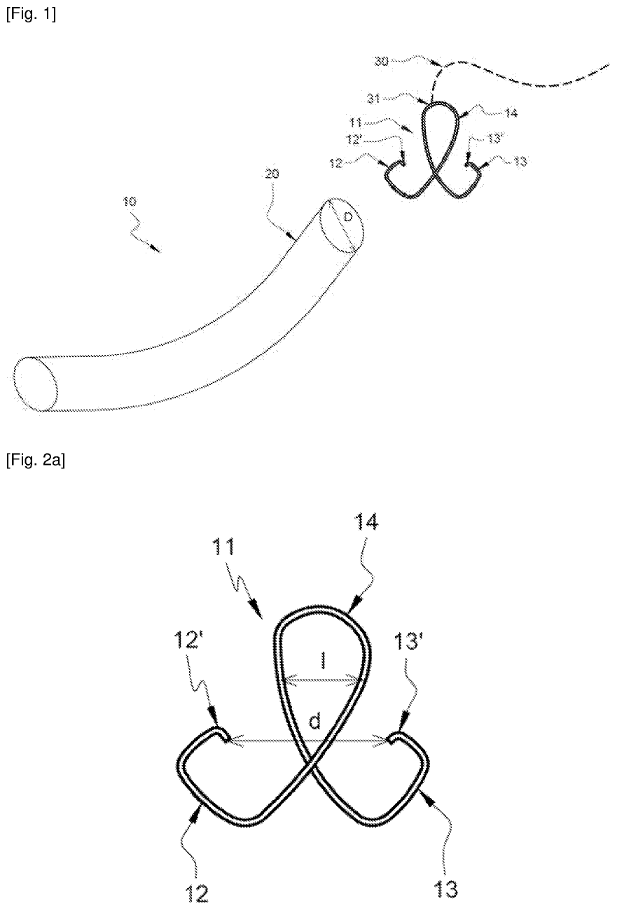

FIG. 1 illustrates the closing device 10 according to an embodiment of the invention.

In reference to FIG. 1, the closing device 10 comprises closing means 11 configured to ensure the closing of the perforation 21.

As can be seen in FIG. 1, the closing means 11 have a central portion 14 arranged between a first end 12 and a second end 13. The central portion 14 is presented in the form of a loop. The first end 12 has a first hooking means 12′ and the second end 13 has a second hooking means 13′. Advantageously, the hooking means 12′, 13′ are arranged symmetrically on either side of the central portion 14. Each hooking means 12′, 13′ has the form of a hook oriented in the direction of the central portion 14, which is intended to be anchored on a border of the perforation 21 at the internal part 3 of the wall 2 of the cavity 1. Furthermore, each end 12, 13 of the closing means 11 forms a bend between each hooking means 12′, 13′ and the central portion 14. Advantageously, the closing means 11 are formed from one same wire such that the loop is formed by crossing the two strands of said wire.

Moreover, the closing means 11 are made of a shape memory material, i.e. that the material has the capacity to keep an initial shape in memory and to return to its initial shape, even after having been deformed. Advantageously, the closing means 11 are made of a nickel-titanium alloy material, as this material has a good elasticity, as well as a certain rigidity. In an embodiment variant, the closing means 11 are made of a resorbable material. In addition, the closing means 11 can have an antithrombogenic and/or antiproliferative and/or procoagulant and/or prothrombogenic coating. In addition, the closing means 11 preferably have a form chosen so as to not cause lesions in the wall 2 of the cavity 1. Also, the closing means 11 have rounded angles, as well as a collected shape. Moreover, the form of the closing means 11 can vary in order to obtain the best resistance with a smaller volume and the least aggression for the wall 2 of the cavity 1.

In addition, the closing means 11 have a structure allowing a variation in the relative position of the hooking means 12′, 13′ with respect to one another according to the state of compression of the central portion 14.

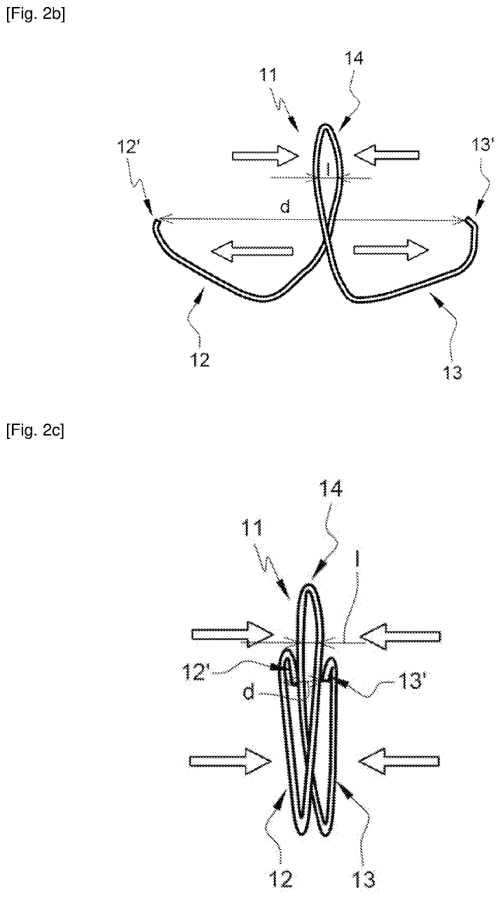

FIGS. 2a, 2b and 2c illustrate the closing means 11 in different positions.

In a rest position illustrated in FIGS. 1 and 2a, the central portion 14 and the ends 12, 13 of the closing means 11 suffer no deformation. The closing means 11 are thus in a relaxed state, i.e. at rest. In this position, the width I of the loop is, for example, between 2 and 5 mm. Furthermore, the distance d between the hooking means 12′, 13′ is for example between 1 and 2 mm.

In the intermediate position illustrated in FIG. 2b, the central portion 14 is in a compressed state. In particular, the central portion 14 is laterally compressed which means here an extension of the loop and therefore a decrease of its width I. In other words, the width I of the loop in the intermediate position is less than the width I of the loop in the rest position. The width I of the loop in the intermediate position is, for example, between 0.1 and 2 mm. Moreover, the compression of the central portion 14 leads to the expansion of the ends 12, 13 of the closing means 11, which extend on either side of the central portion 14. The expansion of the ends 12, 13 of the closing means 11 leads to an increase of the distance d between the hooking means 12′, 13′. Thus, in the intermediate position, the distanced between the hooking means 12′, 13′ is greater than the distance d between the hooking means 12′, 13′ when they are in the rest position. The distance d between the hooking means 12′, 13′ in the intermediate position is, for example, between 1 and 10 mm.

In the compression position illustrated in FIG. 2c, the central portion 14 and the ends 12, 13 of the closing means 11 are in a compressed state. In particular, the central portion 14 and the ends 12, 13 of the closing means 11 are laterally compressed. As explained above, the lateral compression of the central portion 14 leads to an extension of the loop and therefore a decrease of the width I of the loop. In other words, the width I of the loop in the compression position is less than the width I of the loop in the rest position. The width I of the loop in the compression position is, for example, between 0.1 and 2 mm. In addition, the lateral compression of the ends 12, 13 of the closing means 11 leads to a deformation of the ends 12, 13 of the closing means 11 which are folded against the strands of the loop. Thus, the distance d between the hooking means 12′, 13′ in the compression position is less than the distance d between the hooking means 12′, 13′ in the rest position. The distance d between the hooking means 12′, 13′ in the compression position is, for example, between 0.1 and 2 mm.

In the closing device 10 illustrated in FIGS. 1, 3 to 9, the compression of the closing means 11 is ensured by a guide channel 20 which also allows to guide the closing means 11 into the cavity 1, through the perforation 21.

According to the embodiment illustrated in FIGS. 1, 3 to 9, the guide channel 20 is formed by a pipe, the diameter D of which is adapted to the dimensions of the perforation 21 so as to ensure its insertion in said perforation 21. Advantageously, the guide channel 20 is made from a deformable material, for example, polyurethane, in order to be able to be inserted deformed into the cavity 1 and thus avoid causing lesions in the wall 2 of the cavity 1. Furthermore, the diameter D of the guide channel 20 is chosen so as to ensure the compression of the closing means 11 when the latter are housed in the guide channel 20. The diameter D of the guide channel 20 is, for example, between 0.1 and 50 mm.

Moreover, the movement of the closing means 11 in the guide channel 20 is ensured by movement means 30. Advantageously, the movement means 30 are formed by a wire, for example, made of polypropylene, an end 31 of which is fixed to the central portion 14 of the closing means 11, while the other end is kept outside of the body of the patient, in order to be able to be controlled by a practitioner from the outside of the cavity 1.

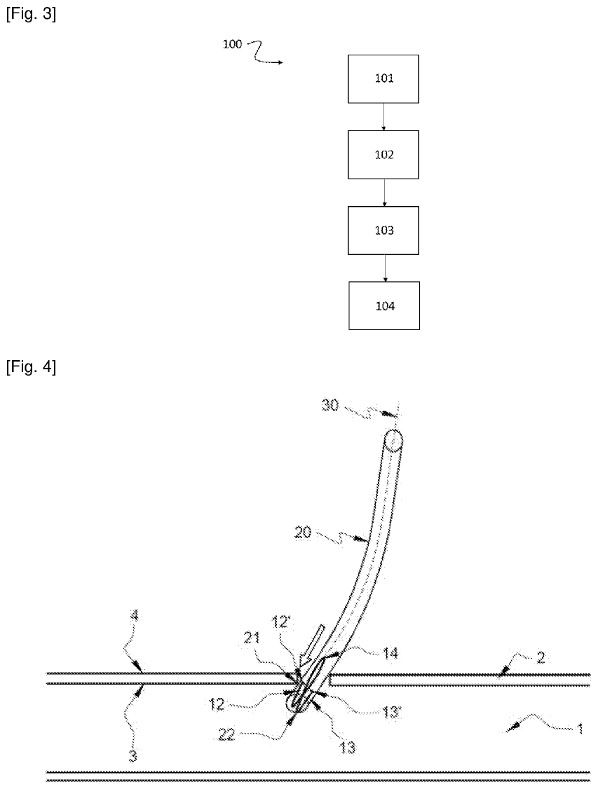

FIG. 3 is a schematic representation in the form of blocks which illustrate the steps of a method for closing 100 a perforation 21, by means of the closing device 10 of FIG. 1. To improve its understanding, the closing method 100 will be described in reference to FIGS. 4 to 9.

In a step 101 illustrated in FIG. 4, the end 22 of the guide channel 20 is inserted in the cavity 1, through the perforation 21. As can be seen in FIG. 4, during the insertion step 101, the closing means 11 are compressed inside the guide channel 20. This is the compression position described above in reference to FIG. 2c. Advantageously, during the insertion step 101, the closing means 11 are positioned close to the end 22 of the guide channel 20 thanks to the movement means 30.

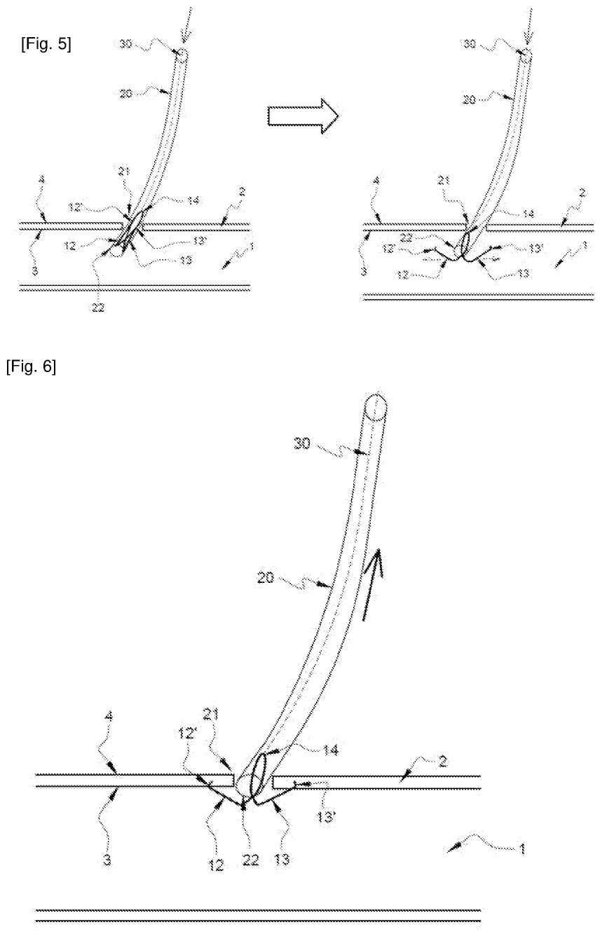

Then, in a step 102 illustrated in FIG. 5, the closing means 11 are partially deployed in the cavity 1. Indeed, in this step 102, the ends 12, 13 of the closing means 11 are deployed outside of the guide channel 20 while the central portion 14 of the closing means 11 is kept in a compressed state inside the guide channel 20. The closing means 11 are thus in the intermediate position described above in reference to FIG. 2b. To do this, the ends 12, 13 of the closing means 11 are moved in the direction of the cavity 1.

At the outlet of the guide channel 20, the hooking means 12, 13 thus pass from a compressed state to a relaxed state leading to the spreading of the hooking means 12′, 13′ from one another. The distance d between the hooking means 12′, 13′ when the closing means 11 are in the intermediate position is thus greater than the distance d between the hooking means 12′, 13′ when the closing means 11 are in the rest position. Advantageously, the distanced between the hooking means 12′, 13′ in this position is greater than the diameter of the perforation 21.

Then, in a step 103 illustrated in FIG. 6, the movement means 30 and the guide channel 20 are partially removed from the cavity 1, through the perforation 21, until the hooking means 12′, 13′ are hooked on the borders of the perforation 21, at the internal part 3 of the wall 2 of the cavity 1. In this step 103, the central portion 14 of the closing means 11 is kept compressed in the guide channel 20.

Subsequently, in a step 104, the guide channel 20 is fully removed from the cavity 1, which leads to the deployment of the central portion 14. Indeed, insofar as the hooking means 12, 13 are hooked to the borders of the perforation 21, at the internal part 3 of the wall 2 of the cavity 1, the hooking means 12, 13 form an abutment preventing the closing means 11 from being extracted from the cavity 1. This is the rest position described in reference to FIG. 2a. The central portion 14 is thus positioned in the perforation 21, mounted between the cavity 1 and the outside of the cavity 1.

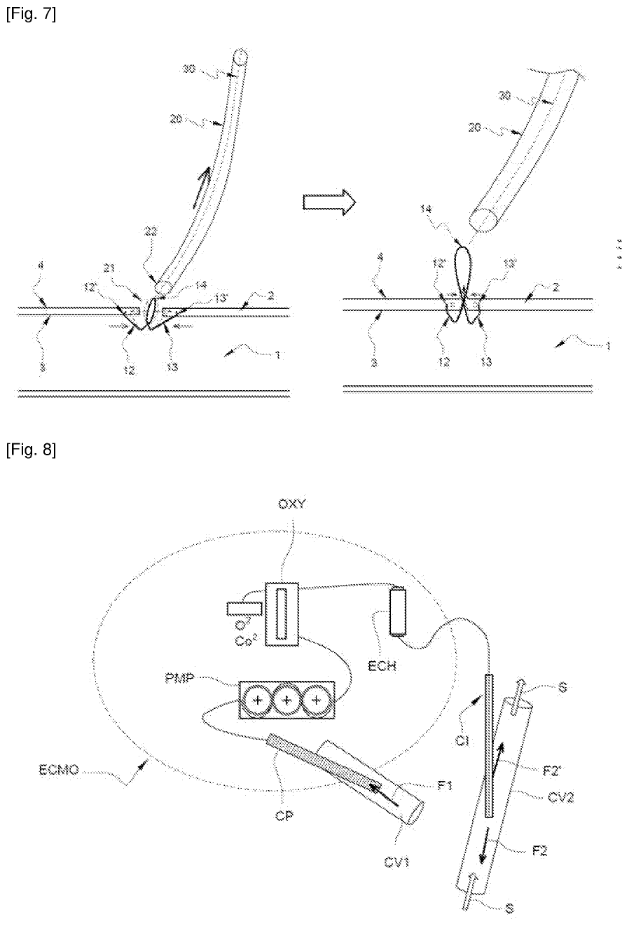

As can be seen in FIG. 7, which illustrates the passage from the intermediate position to the rest position during the removal of the guide channel 20, the central portion 14 passes from a compressed state to a relaxed state, i.e. at rest. The passage from the compressed state to the relaxed state leads to an increase of the width I of the loop and a decrease of the distance d between the hooking means 12′, 13′. The decrease of the distance d between the hooking means 12′, 13′ leads to the alignment of the borders surrounding the perforation 21, as the hooking means 12′, 13′ are anchored on the borders of the perforation 21. The borders are thus flattened against one another, which leads to the closing of the perforation 21. During the closing of the perforation 11, the central portion 14 of the closing means 11 is kept outside of the cavity 11 while the hooking means 12, 13 are kept inside the cavity 11, anchored on the internal wall 3 of the wall 2 of the cavity 1. Advantageously, the movement means 30 are then disconnected from the central portion 14. When the movement means 30 are formed by a wire fixed to the central portion 14, said wire is, for example, cut, by means of scissors or a wire cutter, just underneath the central portion 14.

Moreover, the invention relates to a catheter introducer (not illustrated) in a cavity in a human or animal body, through a perforation. The introducer has a conduit for the passage of the catheter, as well as a closing device identical to the closing device 10 described in reference to FIG. 1. Advantageously, the guide channel of the closing device is formed by the conduit of the introducer. In an embodiment variant, the closing device comprises a guide channel positioned inside the conduit of the introducer, in order to ensure the guiding of the closing means.

The invention also relates to a cannula comprising at least one closing device 10 according to the invention. Advantageously, the cannula according to the invention is an injection cannula or a sampling cannula used in an extracorporeal membrane oxygenation system, known as ECMO.

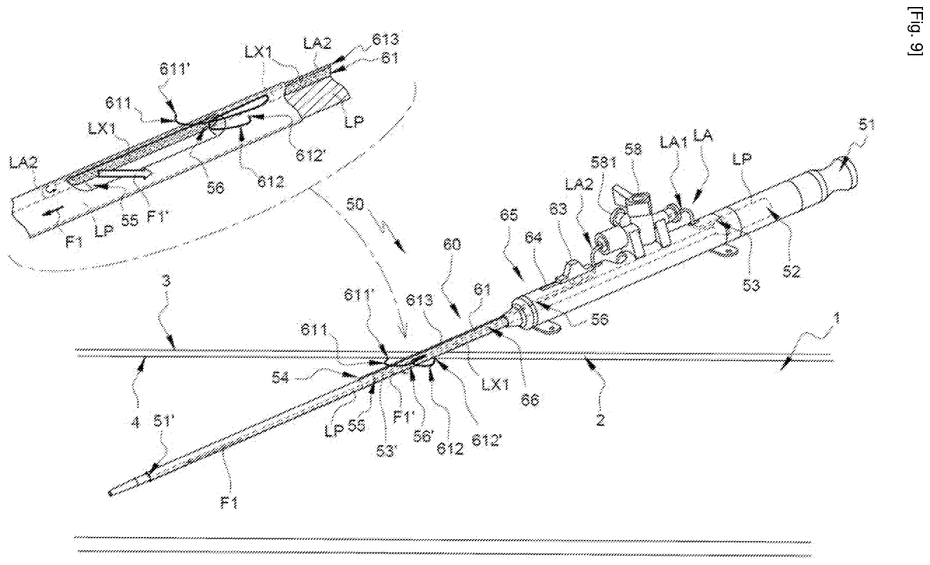

In a known manner and as represented schematically in FIG. 8, an ECMO system has different components, in particular a sampling cannula CP, a pump PMP, an oxygenator OXY, an injection cannula Cl. Thus, the low-oxygen blood flow F1 is sampled in a cavity CV1, by the sampling cannula CP, then channelled to the oxygenator OXY by means of the pump PMP and finally reinjected into a cavity CV2 by means of the injection cannula Cl. The injection cavity CV2 can be identical or a cavity different or identical to the sampling cavity CV1.

Below in the description, it will be recognised that the closing device 10 is integrated with an injection cannula 50 of an ECMO system. Naturally, the closing device 10 can be integrated with any cannula intended to be inserted into a cavity of a human or animal body, such as a sampling cannula of the ECMO system.

Below in the description, it will be recognised that the fluid to be injected into a cavity of the body is blood. In addition, the physiological flow direction S of blood in a vessel, an organ, is called “anterograde flow direction” of a fluid. Furthermore, the opposite physiological flow direction S of blood in a vessel or an organ is called “retrograde flow direction” of a fluid. The baseline is therefore taken with respect to the physiology of the human or animal body. By extension of language and for clarity of the description of the embodiment, the flow direction of the main lumen LP will be called: the “retrograde direction”, and the flow direction of the reperfusion ancillary lumen LA will be called: the “anterograde direction”, each being considered at the outlet of the injection cannula when their direction is mentioned.

Moreover, the terms “upstream” and “downstream” are defined in the present text with respect to the practitioner who handles the injection cannula 50.

Finally, “F2” and “F2′” mean, a treated blood flow, i.e. oxygenated and decarboxylated.

FIG. 9 illustrates a perspective view of an injection cannula 50 according to a first embodiment of the invention, when it is inserted into a perforation 21. The perforation 21 corresponds to the insertion point of the injection cannula 50 in the cavity 1.

As can be seen in FIG. 9, the injection cannula 50 has an inlet 51 capable of engaging with a connecting orifice (not represented) coming from a component of the ECMO, in order to receive the treated blood flow. Furthermore, the injection cannula 50 has an outlet 51′ intended to be inserted into the cavity 1.

Advantageously, the injection cannula 50 is made from a deformable material, for example, polyurethane, possibly heparinised. Thus, the injection cannula 50 can be deformed during its introduction into the cavity 1 so as to be able to be inserted transversally into the wall 2 of the cavity 1 to avoid causing lesions in the wall 2 of the cavity 1. Furthermore, as can be seen in FIG. 9, the injection cannula 50 is partially introduced into the cavity 1 such that a part of the injection cannula 50 remains outside of the body of the patient and thus, that it can be handled by a practitioner.

Furthermore, according to the embodiment of FIG. 9, the injection cannula 50 has a main lumen LP, a reperfusion ancillary lumen LA, as well as a first auxiliary lumen LX1 delimiting three spaces of the injection cannula 50.

The main lumen LP ensures the injection of the blood flow F2 coming from the inlet 51 of the injection cannula 50 into the cavity 1, in a first direction, here a retrograde direction. To this end, the main lumen LP extends along the injection cannula 50 between an inlet 52 and the outlet 51′ of the injection cannula 50 which forms the outlet of the main lumen LP. The inlet 52 of the main lumen LP is arranged downstream from the inlet 51 of the injection cannula 50, in order to receive the blood flow F2. The blood flow F2 is then discharged through the outlet 51′ of the injection cannula 50.

Moreover, the main lumen LP has a circular cross-section and has a diameter which varies between its inlet 52 and its outlet 51′. Furthermore, the diameter of the main lumen LP at its outlet 51′ is smaller than at its inlet 52 such that the part of the injection cannula 50 intended to be inserted into the cavity 1 is adapted to the dimensions of said cavity 1. Advantageously, the diameter of the main lumen LP is between 10F and 21F, i.e. between 3.3 and 7 mm. Naturally, the diameter of the main lumen LP can be constant along the main lumen LP. Furthermore, the main lumen LP could absolutely have a cross-section other than circular, for example, an oval, elliptic cross-section.

The reperfusion ancillary lumen LA ensures the injection of some of the blood flow F2′ into the cavity 1, in a second direction, here an anterograde direction. To this end, the reperfusion ancillary lumen LA has an inlet 53 in fluid communication with the main lumen LP. In particular, the inlet 53 of the reperfusion ancillary lumen LA is arranged downstream from the inlet 52 of the main lumen LP, in order to capture some of the blood flow F2′ coming from the main lumen LP. In an embodiment variant, the inlet 53 of the reperfusion ancillary lumen LA is not in fluid communication with the main lumen LP. In this case, the reperfusion ancillary lumen LA captures some of the blood flow F2′ directly from the inlet 51 of the injection cannula 50. Furthermore, as can be seen in FIG. 9, a portion of the reperfusion ancillary lumen LA extends parallel to the main lumen LP.

Moreover, the reperfusion ancillary lumen LA has a curved portion 53 allowing to modify the retrograde flow direction of the blood flow F2′ captured by the reperfusion ancillary lumen LA. The curved portion 54 allows the blood flow F2′ captured to flow in an anterograde direction. The curved portion 54 forms a circular arc, even a semi-circle allowing to modify the orientation of the fluid F2′ in an anterograde direction, i.e. in a direction opposite the ejection direction of the blood flow F2 into the outlet 51′ of the main lumen LP.

The reperfusion ancillary lumen LA furthermore has an outlet 53′ leading into a side opening 55 arranged in the injection cannula 50 so as to discharge the blood flow F2′ flowing in an anterograde direction into the cavity 1.

The reperfusion ancillary lumen LA, also with a circular cross-section, has, different from the main lumen LP, a constant diameter over the whole of its length. According to an embodiment example, the diameter of the reperfusion ancillary lumen LA has a diameter between 20G, i.e. 0.8 mm, and 6F, i.e. 2 mm. Naturally, the diameter of the reperfusion ancillary lumen LA could vary between the inlet 53 and the outlet 53′ of the reperfusion ancillary lumen LA. It is noted that it is possible to make the ratio of the cross-sections of the lumens LA and LP vary, in order to control the speed and the ejection rate of the blood flow F2, F2′ in the cavity 1 in both directions, i.e. anterograde and retrograde. Furthermore, the reperfusion ancillary lumen LA could have a cross-section other than circular, for example, oval, elliptic.

In an embodiment variant not illustrated, the reperfusion ancillary lumen LA is arranged inside the main lumen LP such as described in patent application FR3058642. In this case, the outlet 53′ of the reperfusion ancillary lumen LA also leads into the side opening 55 arranged in the injection cannula 50. In an embodiment variant not illustrated, the outlet 53′ of the reperfusion ancillary lumen LA is equipped with an electromechanical device for measuring the flow connected to an electronic display located at the handle, i.e. at the inlet 51, of the injection cannula 50.

Moreover, as can be seen in FIG. 9, a drain valve 58 is connected to the reperfusion ancillary lumen LA, in order to ensure its draining. In particular, the drain valve 58 is a three-way valve in which two ways are connected to two portions LA1, LA2 of the reperfusion ancillary lumen LA while a way forms an outlet 581. It is noted that the part of the injection cannula 50 having the drain valve 58 is intended to be kept outside of the human or animal body.

The drain valve 58 allows the opening of two ways and the closing of the third way, simultaneously. Thus, when the outlet 581 of the drain valve 58 is closed, the blood flow F2′ circulating in the reperfusion ancillary lumen LA can pass from the inlet 53 of the reperfusion ancillary lumen LA to the outlet 53′ of the reperfusion ancillary lumen LA. Furthermore, when the outlet 581 of the drain valve 58 is open, the blood flow F2′ cannot pass from the inlet 53 of the reperfusion ancillary lumen LA to the outlet 53′ of the reperfusion ancillary lumen LA.

In a first position of the drain valve 58, called anterograde or control drain of the flow, the blood flow F2′ circulates in the reperfusion ancillary lumen LA, upstream to downstream, i.e. from the inlet 53 of the reperfusion ancillary lumen LA to the outlet 281 of the drain valve 58.

In a second position of the drain valve 58, called retrograde or control drain of the backflow, the blood flow F2′ circulates in the reperfusion ancillary lumen LA from downstream to upstream, i.e. from the outlet 53′ of the reperfusion ancillary lumen LA to the outlet 581 of the drain valve 58. It is noted that the blood flow F2′ circulating in the reperfusion ancillary lumen LA from downstream to upstream can be generated during the introduction of the injection cannula 50 into the cavity 1.

Advantageously, the drain valve 58 allows to ensure that the outlet 53′ of the reperfusion ancillary lumen LA is correctly positioned in the cavity 1. To do this, the flow and the backflow between the outlet 53′ of the reperfusion ancillary lumen LA and the outlet 581 of the drain valve 58 are tested by using a syringe connected to the outlet 581 of the drain valve 58, in order to respectively inject or suction a fluid between the outlet 53′ of the ancillary lumen LA and the outlet 581 of the drain valve 58.

Advantageously, the injection cannula 50 comprises a first closing device 60, allowing to ensure the closing of the perforation 21 corresponding to the insertion point of the injection cannula 50, when the injection cannula 50 is removed from the cavity 1.

The first closing device 60 comprises closing means 61 identical to the closing means 11 described in reference to FIGS. 2a, 2b and 2c. In order to ensure the guiding of the closing means 61 of the first closing device 60, the closing means 61 are housed in the first auxiliary lumen LX1. In other words, the guiding and the compression of the closing means 61 are here ensured by the first auxiliary lumen LX1. In an embodiment variant not illustrated, the first closing device 60 comprises a guide channel placed inside the first auxiliary lumen LX1, in order to ensure the guiding and the compression of the closing means 61.

The first auxiliary lumen LX1 extends parallel to the main lumen LP and has an inlet 56 and an outlet 56′. Advantageously, the first auxiliary lumen LX1 is arranged in a peripheral portion of the injection cannula 50, which can be the wall of the main lumen LP. Furthermore, the outlet 56′ of the first auxiliary lumen LX1, arranged upstream from the outlet 53′ of the reperfusion ancillary lumen LA leads into the side opening 55 arranged in the injection cannula 50. In an embodiment variant, the outlet 56′ of the first auxiliary lumen LX1 and the outlet 53′ of the reperfusion ancillary lumen LA do not lead into the same opening of the injection cannula 50.

Furthermore, the first auxiliary lumen LX1 has a circular cross-section and has a constant diameter above the whole of its length, for example, a diameter between 0.1 and 5 mm. In an embodiment variant, the diameter of the first auxiliary lumen LX1 varies between the inlet 56 and the outlet 56′ of the first auxiliary lumen LX1. It is furthermore noted, that the first auxiliary lumen LX1 can have a cross-section other than circular, for example, elliptic.

The movement of the closing means 61 inside the first auxiliary lumen LX1, then its deployment into the cavity 1 are remotely controlled by movement means 65. In particular, the movement means 65 according to the embodiment of FIG. 9 have a connecting element 66 fixed to the central portion 14 and a pushbutton 63 adapted to slide along a longitudinal groove 64 arranged in the injection cannula 50. The movement, from upstream to downstream, of the pushbutton 63 in the longitudinal groove 64 leads to the movement of the connecting element 66 and therefore of the closing means 61 in the same direction. In particular, during the movement of the pushbutton 63 from upstream to downstream, the latter comes into contact with an end of the connecting element 66, then pushes the connecting element 66 and therefore the closing means 61 in the direction of the cavity 1. At the outlet 56′ of the first auxiliary lumen LX1, the closing means 61 are deployed at least partially in the cavity 1. In an embodiment variant, the deployment of the closing means 61 is done by means of an electric motor located at the handle of the injection cannula 50, under the control of a switch, also located at said handle.

The method for closing the perforation 21 by means of the first closing device 60 of the injection cannula 50 is similar to the closing method 100 described above.

Thus, when the injection cannula 50 is removed from the cavity 1, the closing means 61 housed in the first auxiliary lumen LX1 in a compression position, are pushed in the direction of the cavity 1 by the movement means 30 such that only the ends 611, 612 of the closing means 61 are deployed in the cavity 1, in a relaxed state. The closing means 61 are thus in the intermediate position. Advantageously, the position of the closing means 61 with respect to the injection cannula 50 is blocked by the pushbutton 63 such that when the injection cannula 50 is removed by a few centimetres, the hooking means 611′, 612′ of the ends 611, 612 are anchored on the borders of the perforation 21, at the internal part 3 of the wall 2 of the cavity 1. In this position, the closing means 61 form anchoring means allowing to block in position the injection cannula 50 in the cavity 1. Thus, in the intermediate position, the hooking means 611, 612 prevent the removal of the injection cannula 50 from the cavity 1.

Once the hooking means 611, 612 are anchored in the borders of the perforation 21, the injection cannula 50 is removed such that the central portion 613 of the closing means 61 is deployed outside of the first auxiliary lumen LX1 to return to its rest position. The deployment of the central portion 613 leads to the alignment of the hooking means 611′, 612′, and thus the alignment of the borders of the perforation 21 until it is closed.

Advantageously, the closing means 61 have a radiopaque or echo-opaque marking adapted to ensure an identification of the closing means 61 by radiography or echography. According to another embodiment, the identification marking is made on the outlet 56′ of the auxiliary lumen LX. In another embodiment variant, the closing means 61 and the outlet 56′ of the auxiliary lumen LX are marked.

Moreover, in order to ensure an effective reperfusion of the cavity 1, it is essential that the outlet 53′ of the reperfusion ancillary lumen LA is positioned in the cavity 1 such that the blood flow F2′ flowing in an anterograde direction is ejected right in full light of the cavity 1. In other words, the outlet 53′ of the reperfusion ancillary lumen LA must not be positioned so as to face a wall 2 of the cavity 1.

Also, the outlet 56′ of the auxiliary lumen LX is positioned at a predetermined distance d from the outlet 53′ of the reperfusion ancillary lumen LA such that, when the injection cannula 50 is blocked in position in the cavity 1 by the hooking means 611, 612, the blood flow F2′ which circulates in the reperfusion ancillary lumen LA is discharged correctly into the cavity 50, i.e. in the anterograde direction. Advantageously, the predetermined distance between the outlet 56′ of the auxiliary lumen LX and the outlet 53′ of the reperfusion ancillary lumen LA is between 0.1 and 500 mm.

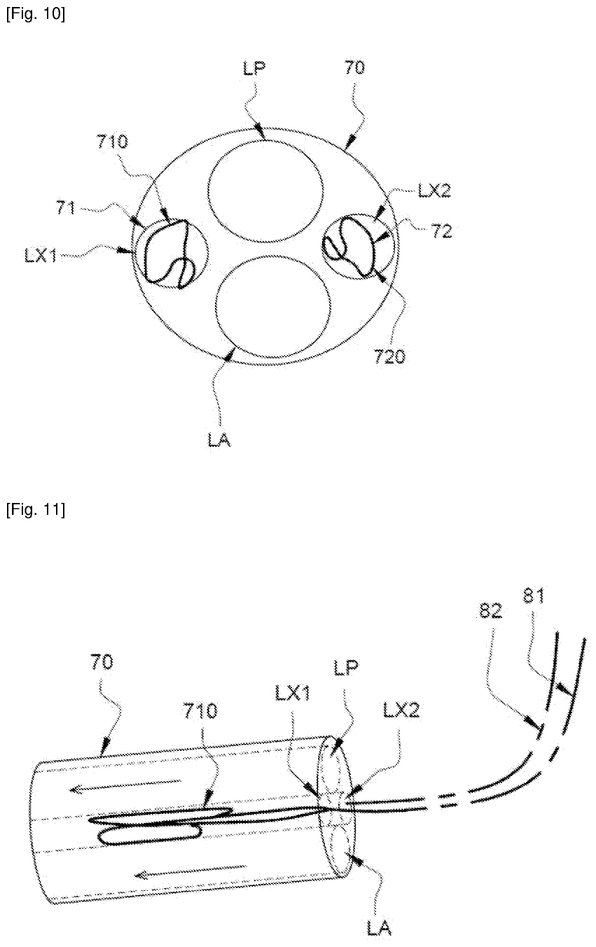

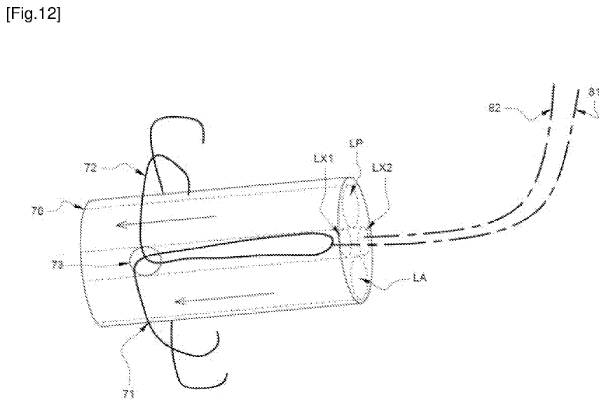

FIGS. 10, 11 and 12 illustrate an injection cannula 70 according to a second embodiment.

The injection cannula 70 is broadly identical to the injection cannula 50 according to the first embodiment, the only difference being that it comprises, in addition to a first auxiliary lumen LX1 and a first closing device 71, a second auxiliary lumen LX2 and a second closing device 72.

The first closing device 71, positioned inside the first auxiliary lumen LX1, comprises closing means 710, as well as a connecting element 81 fixed to the central portion (which cannot be seen) of the closing means 710 of the first closing device 71. The second closing device 72, positioned inside the second auxiliary lumen LX2, comprises, in the same way as the first closing device 71, closing means 720, as well as a connecting element 82 fixed to the central portion (which cannot be seen) of the closing means 720 of the second closing device 72. Advantageously, the second auxiliary lumen LX2 is identical to the first auxiliary lumen LX1 and is arranged in a peripheral portion of the injection cannula 70 opposite the peripheral portion in which the first auxiliary lumen LX1 is arranged. The outlet 73 of the first auxiliary lumen LX1 and the outlet of the second auxiliary lumen LX2 are, preferably, arranged at the same level of the injection cannula 70.

The invention also relates to an ECMO system having the injection cannula 50 according to the first embodiment of the injection cannula 70 according to the second embodiment of the invention.

1. Device for closing (10) a perforation (21) in a wall (2) of a cavity (1) in a human or animal body, said wall (2) having an internal part (3) and an external part (4), characterised in that the closing device (10) has closing means (11) comprising:
a central portion (14),
a first end (12) having a first hooking means (12′) and a second end (13) having a second hooking means (13′), the two ends (12, 13) being arranged on either side of the central portion (14), each hooking means (12′, 13′) being configured to be anchored on a border of the perforation (21) at the internal part (2) of the wall (2) of the cavity (1),
the closing means (11) being made of a shape memory material and having a structure allowing a variation in the relative position of the hooking means (12′, 13′) with respect to one another according to the state of compression of the central portion (14) such that when the central portion (14) is in a compressed state, the distance (d) between the hooking means (12′, 13′) increases and that when the central portion (14) passes from a compressed state to a relaxed state, the distance (d) between the ends (12′, 13′) of the hooking means (11) decreases, which leads to the alignment of the borders of the perforation (21) and therefore the closing of the perforation (21).

2. Closing device (10) according to the preceding claim, characterised in that it has a guide channel (20) having an end (22) adapted to be positioned in the cavity through the perforation (21), the guide channel (20) being configured to receive the closing means (11) and to ensure the compression of said closing means (11).

3. Closing device (10) according to the preceding claim, characterised in that the closing means (11) are configured to adopt:
a compression position in which the central portion (14) and the hooking means (12, 13) are in a compressed state in the guide channel (20),
a rest position in which the central portion (14) and the hooking means (12, 13) are outside of the guide channel (20) and in a relaxed state,
an intermediate position in which the central portion (14) is in a compressed state in the guide channel (20) while the hooking means (12, 13) are outside of the guide channel (20) in a relaxed state, the distance (d) between the hooking means (12′, 13′) in the intermediate position being greater than the distance (d) between the hooking means (12′, 13′) in the rest position.

4. Closing device (10) according to any one of claims 2 to 3, characterised in that it has movement means (30) configured to ensure the movement of the closing means (11) inside the guide channel (20).

5. Closing device (10) according to the preceding claim, characterised in that the movement means (30) are removably fixed to the central portion (14) of the closing means (11).

6. Closing device (10) according to any one of the preceding claims, characterised in that the central portion (14) has the form of a loop.

7. Closing device (10) according to any one of the preceding claims, characterised in that the first hooking means (12) and the second hooking means (13) each have the form of a hook.

8. Closing device (10) according to any one of the preceding claims, characterised in that the closing means (11) are made of a resorbable material.

9. Closing device (10) according to any one of claims 1 to 7, characterised in that the closing means (11) are made from a nickel-titanium alloy wire.

10. Catheter introducer in a cavity (1) of a human or animal body, through a perforation, having a conduit configured for the passage of the catheter, characterised in that it has a closing device (10) according to any one of the preceding claims, configured to be positioned inside the conduit and to ensure the closing of the perforation by deploying the closing means (11) of the closing device (10) in the cavity during the removal of the introducer from the perforation.

11. Cannula (50, 70) for injecting or sampling a fluid in a perforation (21) of a wall (2) of a cavity (1) in a human or animal body, having a main lumen (LP) defining a first space of the cannula (50) for the circulation of the fluid, characterised in that the cannula (50) furthermore has:
a first auxiliary lumen (LX1) defining a second space of the cannula (50) and having an outlet (56′) leading into the cavity (50),
a first closing device (10, 71) according to any one of claims 1 to 9, configured to be positioned inside the first auxiliary lumen (LX1) and to ensure the closing of the perforation (21) by deploying the closing means (61, 710) of the first closing device (10, 70) in the cavity (1) during the removal of the cannula (50) from the perforation (21).

12. Cannula (50, 70) according to the preceding claim, characterised in that it has:
a second auxiliary lumen (LX2) defining a third space of the cannula (50, 70) and having an outlet leading into the cavity (1),
a second closing device (72) according to any one of claims 1 to 10, configured to be positioned inside the second auxiliary lumen (LX2).

13. Cannula (50, 70) according to any one of claims 11 to 12, characterised in that the first auxiliary lumen (LX1) and/or the second auxiliary lumen (LX2) is/are arranged in a peripheral portion of the cannula (50, 70).

14. Cannula (50, 70) according to any one of claims 11 to 13, characterised in that the closing means (61, 71, 72) form means for anchoring the cannula (50) in the cavity (1) when the closing means (61, 71, 72) are in an intermediate position.

15. Extracorporeal membrane oxygenation system having:
a sampling cannula (CP) to receive a low-oxygen fluid;
a pump (PMP) to ensure the pumping of the low-oxygen fluid coming from the sampling cannula;
an oxygenator (OXY) to ensure the oxygenation of the low-oxygen fluid coming from an outlet of the pump (PMP);
an injection cannula (Cl) to inject the oxygenated fluid into the cavity (1);
characterised in that the sampling cannula (CP) and/or the injection cannula (Cl) of the system is/are formed by the cannula (50) according to any one of claims 11 to 14.

16. Method for closing a perforation (100), by means of the closing device (10) according to one of claims 1 to 9, characterised in that the method (100) has:
a step (101) of inserting the end (22) of the guide channel (20) in the cavity (1), the closing means (11) are compressed inside the guide channel (20);
a step (102) of partially deploying closing means (11) in the cavity (1), the ends (12, 13) of the closing means (11) are deployed outside of the guide channel (20) while the central portion (14) of the closing means (11) is kept in a compressed state inside the guide channel (20);
a step (103) where the movement means (30) and the guide channel (20) are partially removed from the cavity (1), through the perforation (21), until the hooking means (12′, 13′) are hooked on the borders of the perforation (21) at of the internal part (3) of the wall (2) of the cavity (1), the central portion (14) of the closing means (11) is kept compressed in the guide channel (20);
a step (104) where the guide channel (20) is fully removed from the cavity (1) leading to the deployment of the central portion (14), the hooking means (12, 13) are hooked to the borders of the perforation (21), at of the internal part (3) of the wall (2) of the cavity (1) and form an abutment preventing the closing means (11) from being extracted from the cavity (1).
